(19) United States
(12) Patent Application Publication
(10) Pub. No.: US 2013/0305788 A1
(43) Pub. Date: Nov. 21, 2013

(54) ELASTIC KNITTING THREAD AND MANUFACTURE THEREOF

(71) Applicant: medi GmbH & Co. KG, Bayreuth (DE)

(72) Inventor: Jan ATMANSPACHER, WARMENSTEINACH (DE)

(73) Assignee: medi GmbH & Co. KG, Bayreuth (DE)

(21) Appl. No.: 13/893,997

(22) Filed: May 14, 2013

(30) Foreign Application Priority Data
May 15, 2012 (DE) 10 2012 009582.1

(51) Int. Cl. D02G 3/32; A61F 13/08; D04B 1/26; D02G 3/36

(52) U.S. Cl. CPC D02G 3/32; D02G 3/36; A61F 13/08; D04B 1/26
USPC 66/178 A; 57/225; 57/230; 57/3; 57/15; 66/202

(57) ABSTRACT

Elastic knitting thread, having a core of an elastic material, an inner wrapper surrounding the core and an outer wrapper surrounding the inner wrapper, wherein one yarn is a flat yarn and the other yarn is a textured yarn.

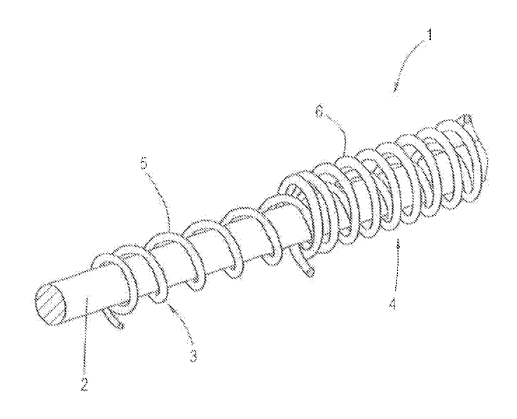

The invention relates to an elastic knitting thread, having a core of an elastic material, an inner wrapper surrounding the core and an outer wrapper surrounding the inner wrapper.

Elastic knitting threads of this type are used in the manufacture of a very wide variety of textiles, one prominent example being the manufacture of stockings, primarily ladies' stockings/pantyhose, but also notably in the form of compression stockings designed to apply a defined level of compression to the leg for medical/therapeutic reasons. One known knitting thread comprises a core of an elastic material, surrounded by an inner wrapper of a first yarn, which in turn is surrounded by an outer wrapper composed of a second yarn. A yarn of this type is known from DE 20 2010 013 665 U1 for example. The two yarns used therein are of differing type. The inner wrapper is formed by a microfiber yarn, while the outer wrapper can be fabricated using an ultrafine yarn or a multifilament yarn. The two yarns used in DE 20 2010 013 665 U1 are notable for a special ability to transport moisture whereby the thread described and a medical compression stocking manufactured therefrom are said to provide particularly good transportation of moisture.

However, compression knits manufactured from a knitting thread of the type referred to at the outset occasionally present the problem of the knit, i.e., the compression stocking for example, frequently being very difficult to don owing to the very high stretch recovery power of the knitting thread, since it is this high stretch recovery power which causes the knit to undergo substantial extension as it is being donned, making donning difficult.

The problem addressed by the invention is accordingly that of specifying an elastic knitting thread which has improved stretch recovery characteristics, so textiles fabricated therefrom, such as compression stockings in particular, are simpler to don.

To solve this problem, the invention provides for an elastic knitting thread of the type referred to at the beginning that one yarn is a flat yarn and the other yarn is a textured yarn.

The invention thus employs two different yarns, of which one is a flat yarn, while the other one is a textured yarn, which is textured in a defined manner in the manufacturing operation or in yarn preparation. A yarn of this type is deformed in the manufacturing operation in a defined manner so that it acquires a slight crimp. Using, then, a flat yarn and a textured yarn to form the two wrappers will result in differing extensibility along the length of the thread. Whereas the textured yarn can be extended relatively strongly owing to its texturing, i.e., crimp, the flat thread is distinctly less extensible. As a consequence, the entire hybrid construction, i.e., the knitting thread, will extend to a distinctly smaller degree than an elastic knitting thread fabricated from two textured yarns. Owing to the reduced stretch recovery characteristics, the final compression knit, i.e., the compression stocking or the like, will have distinctly different extensibility characteristics as a result, so it surprisingly turns out to be distinctly simpler and more convenient to don.

It is preferably the flat yarn which forms the inner wrapper, while the outer wrapper is formed by the textured yarn. Forming the outer wrapper using the textured yarn endows the knit with very pleasant haptics, which are advantageous for a pleasant sense of wearing comfort.

It is preferable to use a preshrunk yarn as textured yarn. This is advantageous because a dyeing operation on the final knit to confer a certain color on the latter, which involves a thermal operation, will cause the textured yarn to shrink only slightly, if at all, while the flat yarn by contrast will undergo somewhat greater shrinkage. This can be used to ensure that the dyeing operation does not lead to major, possibly incalculable changes with regard to the extensibility properties of the knit. It will be appreciated that the flat yarn may also be preshrunk.

Useful yarns include microfiber yarns, multifilament yarns or standard dpf yarns, and the two yarns can be the same or different. That is, the inner wrapper can for example consist of a microfiber yarn and the outer wrapper of a multifilament yarn, although it will be appreciated that even yarns of the same type such as two multifilament yarns for example can be used to form the inner and outer wrappers.

In a particularly advantageous development of the invention, the numbers of turns per meter at which the two yarns twist around the core are different. That is, either the first yarn or the second yarn has more turns per meter than whichever is the other yarn. The extensibility characteristics can be varied in a controlled manner as a result. Depending on whether, then, the flat yarn or the textured yarn is twisted with the higher number of turns per meter, the fraction of the respective yarn will vary per thread meter, and so a difference in the extensibility characteristics of the two yarns will result in a corresponding change of the overall extensibility characteristics. It will be appreciated that it is also possible in principle to twist the two yarns with the same number of turns per meter.

It is preferably the textured yarn, especially when it forms the outer wrapper, which is twisted with a higher number of turns per meter.

The number of turns per meter of the particular yarn should be between 500-2000 T/m. The number of turns per meter is preferably in the range between 600-1500 T/m, especially between 700-1200 T/m and more preferably between 800-1000 T/m. When the two yarns have been twisted with different numbers of turns per meter, the difference should be at least 25 T/m, preferably at least 50 T/m. The difference can also amount to a greater number of turns per meter, for example 100 or 150 T/m, in which case the extensibility characteristics of the thread can also be varied as a result, as will be appreciated.

The core itself preferably consists of elastane, while the yarns consist of nylon-6 or nylon-6,6. It will be appreciated that other materials can also be used to form the core and the yarns respectively.

In addition to the thread itself, the invention provides a textile knit obtained from an elastic knitting thread of the type described. The knit is preferably a compression stocking.

The invention further provides a process for manufacturing an elastic knitting thread of the type described, characterized in that a core of elastic material is overwrapped with an inner wrapper of a first yarn and this inner wrapper is overwrapped with an outer wrapper of a second yarn, wherein one yarn is a fiat yarn and the other yarn is a textured yarn.

It may be preferable to use the flat yarn to form the inner wrapper, while the textured yarn is used to form the outer wrapper. This is advantageous for haptic reasons in particular. At any rate, however, there is nothing to gainsay a converse arrangement.

The textured yarn used is preferably a preshrunk yarn, although the flat yarn may also be preshrunk of course.

Microfiber yarns, multifilament yarns or standard dpf yarns can be used in principle, in which case the two yarns used can be of the same type or different. Preferably, however, yarns of the same type in the form of multifilament yarns are used. The differentiation between the microfiber yarn and multifilament yarn and standard dpf yarn is via the ratio of linear density to the number of filaments. Yarn linear density has the units of dtex, i.e., decitex, where 1 dtex corresponds to a filament weight of g/10 000 m. The number of filaments can likewise be varied. A ratio of linear density/number of threads is ≦1 for a microfiber yarn, >1.0-2.5 for a multifilament yarn and >2.5 for a standard dpf yarn.

In a further development of the invention, the process is characterized in that the two yarns are twisted around the core preferably at different numbers of turns per meter, wherein the respective number of turns per meter is between 500-2000 T/m, preferably between 600-1500 T/m, especially between 700-1200 T/m and more preferably between 800-1000 T/m, Any intermediate value shall be deemed disclosed as essential to the invention. It will be appreciated that the two yarns can also be twisted with the same number of turns per meter.

When the numbers of turns per meter differ they differ by at least 25 Tim, but the difference can also be greater. Preferably, the textured yarn is twisted at a higher number of turns per meter, especially when it forms the outer wrapper.

As regards materials, it is preferable to use a core of elastane and nylon-6 or nylon-6,6 as yarn materials.

Further advantages, features and details of the invention will become apparent from the exemplary embodiment described hereinbelow and also from the drawing.

This in-principle depiction shows an inventive elastic knitting thread 1 consisting of a core 2 of preferably elastane, an inner wrapper 3, twisted directly around the core 2, and also an outer wrapper 4, which surrounds the inner wrapper 3. The inner wrapper 3 in the exemplary embodiment shown is twisted from a flat yarn 5, while the outer wrapper 4 is twisted from a textured yarn 6. The yarns preferably consist of nylon-6 or nylon-6,6. The yarns 5, 6 are preferably two multifilament yarns which, as stated, differ in that yarn 5 is flat while yarn 6 is textured.

It is apparent that the first yarn 5 twists around the core 2 at a lower number of turns per meter than the second yarn 6. For example, the twist level can be 850 T/m for the first yarn and 900 T/m for the second yarn 6. The direction of twist of the two yarns 5, 6 in the illustrative example is the same, namely an S/S twist. Other forms of twisting are also conceivable of course, such as both yarns 5, 6 being twisted into the other direction, i.e., a Z/Z twist or for the two yarns 5, 6 to be twisted into different directions of twist, i.e., an S/Z or a Z/S twist.

As the textured yarn 6 forms the outer wrapper 4 here, the knit fabricated therefrom has very pleasant haptics, which are advantageous for the wearing comfort.

The two yarns 5, 6 further have different extensibility characteristics. The preferably preshrunk textured yarn 6 is distinctly further extensible, since its texturing means that it can be elongated further than the flat yarn 5. The result is reduced stretch recovery power on the part of thread 1, i.e., the thread is altogether somewhat less extensible, which has advantageous repercussions for the knit manufactured therefrom. The donnability of a stocking manufactured from this thread 1 is simplified.

A preferred knitting thread, which is very useful in the manufacture of compression stockings, includes a core composed of 44/45 dtex elastane. The inner wrapper 3 is embodied in a 33-dtex 20-filament flat nylon-6,6 yarn, while the outer wrapper 4 is embodied in a nylon-6,6 yarn at 33 dtex/20 filaments also. The inner yarn 5 has a twist level of 850 T/m, while the outer yarn 6 has a twist level of 900 T/m, for an S/S twist. There is accordingly a twist level difference of 50 T/m. However, this is merely an exemplary embodiment in that the invention shall not be deemed limited thereto. It will be appreciated that other yarns 5, 6 having other extensibility characteristics/filament numbers coupled with other twist levels/directions of twist can also be used.

1. Elastic knitting thread, having a core of an elastic material, an inner wrapper surrounding the core and an outer wrapper surrounding the inner wrapper, characterized in that one yarn is a flat yarn and the other yarn is a textured yarn.

2. Elastic knitting thread according to claim 1, wherein the flat yarn forms the inner wrapper and the textured yarn forms the outer wrapper, or vice versa.

3. Elastic knitting thread according to claim 1, wherein the textured yarn is preshrunk.

4. Elastic knitting thread according to claim 1, wherein the yarns are microfiber yarns, multifilament yarns or standard dpf yarns, while the two yarns are of the same type or different.

5. Elastic knitting thread according to claim 4, wherein both the yarns are multifilament yarns.

6. Elastic knitting thread according to claim 1, wherein the numbers of turns per meter at which the two yarns twist around the core are the same or different.

7. Elastic knitting thread according to claim 6, wherein the textured yarn has a higher number of turns per meter.

8. Elastic knitting thread according to claim 6, wherein the number of turns per meter is between 500-2000 T/m, preferably between 600-4500 T/m, especially between 700-1200 T/m and more preferably between 800-1000 T/m.

9. Elastic knitting thread according to claim 6, wherein different numbers of turns per meter differ by at least 25 T/m.

10. Elastic knitting thread according to claim 6, wherein the core consists of elastane and the yarns consist of nylon-6 or nylon-6,6.

11. Elastic knitting thread according to claim 6, wherein the two yarns are twisted S/S, Z/Z, S/Z or Z/S.

12. Process for manufacturing an elastic knitting thread according to claim 6, wherein a core of elastic material is overwrapped with an inner wrapper of a first yarn and this inner wrapper is overwrapped with an outer wrapper of a second yarn, wherein one yarn is a flat yarn and the other yarn is a textured yarn.

13. Process according to claim 12, wherein the flat yarn is used to form the inner wrapper and the textured yarn is used to form the outer wrapper, or vice versa.

14. Process according to claim 12, wherein the textured yarn used is a preshrunk yarn.

15. Process according to claim 12, wherein the yarns used are microfiber yarns, multifilament yarns or standard dpf yarns, wherein the two yarns used are of the same type or different, wherein yarns of the same type are used in the form of multifilament yarns in particular.

16. Process according to claim 12, wherein the two yarns are twisted around the core at the same or different numbers of turns per meter, wherein the number of turns per meter is between 500-2000 T/m preferably between 600-1500 T/m, especially between 700-1200 T/m and more preferably between 800-1000 T/m.

17. Process according to claim 16, wherein when the numbers of turns per meter differ by at least 25 T/m.

18. Process according to claim 16, wherein the textured yarn is twisted at a higher number of turns per meter.

19. Process according to claim 12 wherein a core of elastane and yarns of nylon-6 and nylon-6,6 are used.

20. Textile knit obtained from an elastic knitting thread according to claim 1.

21. Textile knit according to claim 20, wherein it is a compression stocking.
